(19) United States
(12) Patent Application Publication
(10) Pub. No.: US 2014/0180326 A1
(43) Pub. Date: Jun. 26, 2014

(54) INFLATABLE BALLOON FOR PROTECTING BLOOD VESSEL

(71) Applicant: Empire Technology Development LLC, Wilmington, DE (US)

(72) Inventors: John Mathew Adams, Snohomish, WA (US); DAniel Hawkins, Bellevue, WA (US)

(73) Assignee: Empire Technology Development LLC, Wilmington, DE (US)

(21) Appl. No.: 13/995,497

(22) PCT Filed: Dec. 20, 2012
(86) PCT No.: PCT/US12/71094

§ 371 Date: Jun. 18, 2013

(51) Int. Cl. A61F 2/82; A61B 17/12; A61M 29/02

(52) U.S. Cl. CPC A61F 2/82; A61M 29/02; A61B 17/12136
USPC 606/192

(57) ABSTRACT

Technologies are generally provided for a vascular reinforcement device is provided for preventing compression of a blood vessel in the presence of forces applied externally to the blood vessel. The vascular reinforcement device may include a balloon having a substantially elongated shape which may be placed adjacent to a left renal vein such that when the balloon is in an inflated position, the balloon may prevent compression of the left renal vein against a rigid structure such as the aorta. The balloon may include an anchoring sheath on each end of the balloon for anchoring the inflatable balloon to fascia surrounding the blood vessel to provide stabilization. The balloon may be delivered and positioned in an initial deflated position employing a laparoscopic procedure, and once anchored in position alongside the left renal vein, the balloon may be filled with a sterile solution for inflation.

### BACKGROUND

Unless otherwise indicated herein, the materials described in this section are not prior art to the claims in this application and are not admitted to be prior art by inclusion in this section.

Preeclampsia is a pregnancy induced hypertension that can be associated with proteinuria (an excess of serum proteins in the urine) and edema. Preeclampsia typically occurs in 5-10% of pregnancies, and is characterized by symptoms such as swelling, sudden weight gain, headaches and changes in vision. Preeclampsia can progress to eclampsia, with cerebral symptoms leading to convulsions. The condition is associated with systemic vasospasm wherein arteries throughout the body narrow. This can lead to multi-organ system dysfunction wherein many organs of the body, including the kidneys, brain, eyes, liver, etc., are unable to function normally because of altered blood flow and increased blood pressure. Currently the only effective treatment is delivery of the fetus and placenta. Typically, preeclampsia occurs after 20 weeks gestation (in the late 2nd or 3rd trimester), though it can occur earlier.

While the cause of preeclampsia is still being debated, inadequate blood supply to the placenta, abnormalities in the immune system and maternal endothelial cell dysfunction are suspected to be involved. A theorized cause of preeclampsia is compression of the left renal vein due to increased abdominal pressures caused by the growing uterus and abdomen during pregnancy. Abdominal organs can shift due to the growing uterus and can pin the left renal vein, which passes between the vertebra and the aorta, against the rigid aorta causing the blood pressure within the left renal vein to increase substantially. The increase in blood pressure within the left renal vein activates a biological system in the kidney which in effect causes increased system blood pressure, or hypertension. Other circumstances can also trigger the hypertension symptoms, for example, obesity can cause external forces to be exerted on the left renal vein against the aorta, leading to a higher than normal renal vein pressure resulting in hypertension and preeclampsia symptoms.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

According to some examples, the present disclosure generally describes a vascular reinforcement device for preventing compression of a blood vessel. The vascular reinforcement device may include an inflatable balloon having a substantially elongated shape configured to be placed adjacent to a wall of a blood vessel along a portion of the blood vessel such that when the inflatable balloon is in an inflated position, an external pressure applied to the blood vessel is dampened.

According to other examples, the present disclosure also describes a method of preventing compression of a blood vessel from external bodily tissue forces. The method may include providing a vascular reinforcement device configured to resist external pressures from compressing a blood vessel, the vascular reinforcement device including an inflatable balloon having a substantially elongated shape, and a sheath attached to a first end and a second end of the inflatable balloon for enabling the inflatable balloon to be anchored to fascia surrounding the blood vessel, and positioning the inflatable balloon adjacent to a wall of the blood vessel along a portion of the blood vessel such that when the inflatable balloon is in an inflated position, an external pressure applied to the blood vessel may be dampened.

According to further examples, the present disclosure also describes a system for preventing compression of a vein from external bodily tissue forces. The system includes a surgical delivery tube for providing percutaneous access to an internal area of a body via at least one incision, and an inflatable balloon having a substantially elongated shape, wherein the inflatable balloon may be configured in an initial deflated position for delivery to the internal area of the body via the delivery tube and inflated to an inflated position at the internal area of the body, and a sheath attached to a first end and a second end of the inflatable balloon for anchoring the inflatable balloon to fascia surrounding the blood vessel.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example anatomical layout of the abdomen including the kidneys, left renal vein, inferior vena cava, and the aorta;

FIG. 2 illustrates an arrangement of the left renal vein and renal artery and a schematic of the corresponding blood pressures;

FIG. 3 illustrates the location of the left renal vein where it crosses the aorta and a schematic diagram of the blood pressure when the left renal vein is compressed;

FIG. 4 illustrates an inflatable balloon for protecting the left renal vein from external pressures;

FIG. 5 illustrates an inflatable balloon anchored in position over the aorta and adjacent to the left renal vein;

FIG. 6 illustrates inflation of an inflatable balloon employing a needle and port; and

FIG. 7 illustrates delivery of an inflatable balloon employing laparoscopic tools; all arranged in accordance with at least some embodiments as described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, apparatus, systems, devices, and/or computer program products related to providing a vascular reinforcement device for preventing compression of a vein.

Briefly stated, a vascular reinforcement device is provided for preventing compression of a blood vessel in the presence of forces applied externally to the blood vessel. The vascular reinforcement device may include a balloon having a substantially elongated shape which may be placed adjacent to a left renal vein such that when the balloon is in an inflated position, the balloon may prevent compression of the left renal vein against a rigid structure such as the aorta. The balloon may include an anchoring sheath on each end of the balloon for anchoring the inflatable balloon to fascia surrounding the blood vessel to provide stabilization. The balloon may be delivered and positioned in an initial deflated position employing a laparoscopic procedure, and once anchored in position alongside the left renal vein, the balloon may be filled with a sterile solution for inflation.

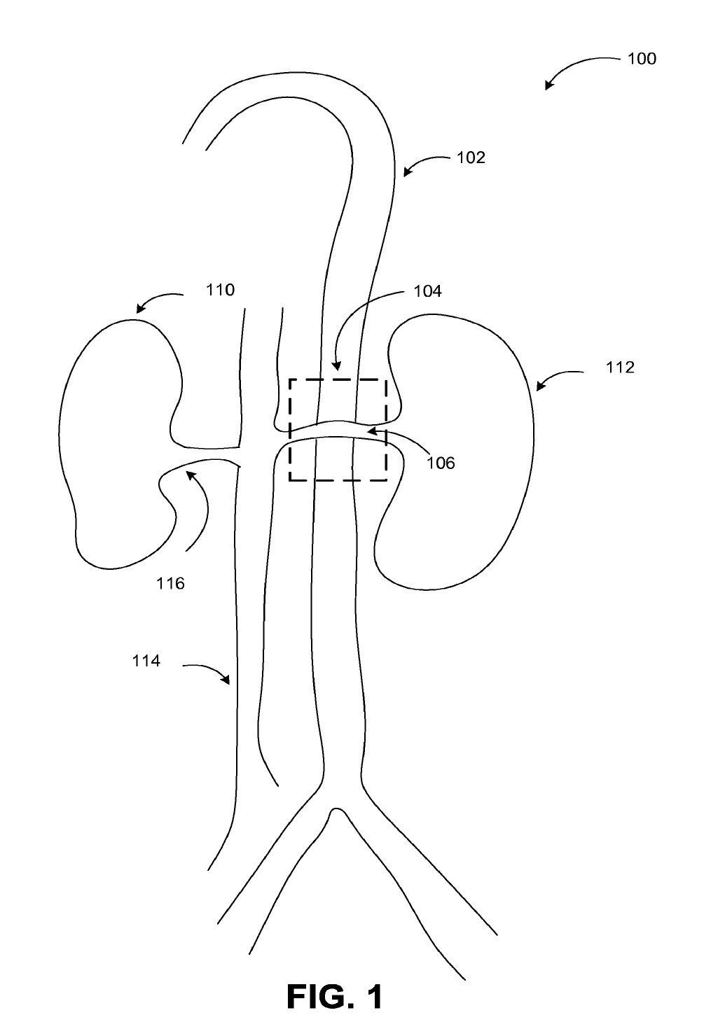

FIG. 1 illustrates an example anatomical layout of the abdomen including the kidneys, left renal vein, inferior vena cava, and the aorta, arranged in accordance with at least some embodiments as described herein. As illustrated in diagram 100, the abdominal cavity includes a right kidney 110, a left kidney 112, inferior vena cava 114, aorta 102, left renal vein 106, and right renal vein 116. The left renal vein 106 connects the left kidney 112 to the inferior vena cava 114 for support blood flow from the left kidney 112 through the inferior vena cava 114 and back to the heart. The left renal vein 106 passes over 104 and is immediately adjacent to the aorta 102. The aorta 102 is a large artery and is at high fluid/blood pressure and has a rigid structure when compared to the compliant vascular structure of a relatively low pressure in the left renal vein 106.

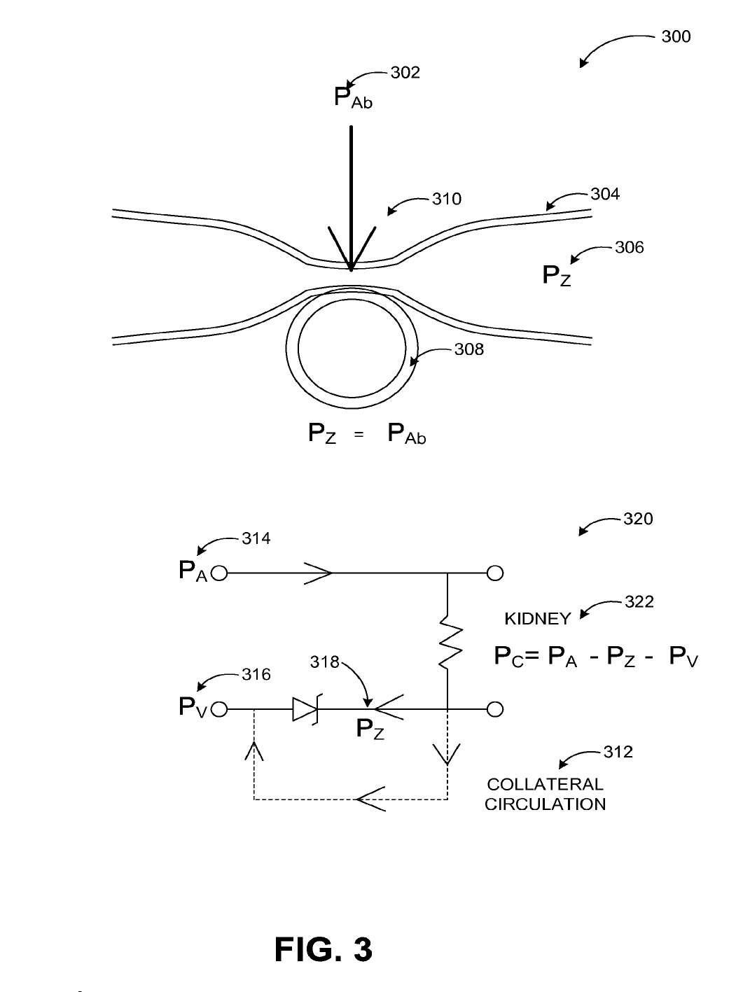

Often times the left renal vein 114 can be subjected to compressive forces within the abdominal area. For example the expansion of tissue and organs in obese persons and the expanding uterus in pregnant persons can cause compression of the left renal vein 114 against the rigid aorta 102 due to increased abdominal pressure and shifting organs. When the left renal vein 114 is compressed against the rigid aorta 102, the left renal vein 114 may distort, such that a diameter of the left renal vein 114 decreases due to the compression against the rigid aorta 102. When the diameter of the left renal vein 114 decreases, the left kidney 112 attempts to maintain a constant blood flow through the left renal vein 114 and the blood pressure within the left renal vein 114 increases on the left side of the aorta, i.e. upstream from the restriction on the left renal vein. The increased blood pressure within the left renal vein 114 is sensed as if it were a reduced aortic pressure by sensors the left kidney 112, as described further in FIG. 3, and in response, the left kidney 112 activates the renin-angiotensin system (RAS). The RAS triggers an increased production of renin enzyme which leads to increased angiotensin II production. Angiotensin II causes the blood vessels within the body to constrict, leading to systemic (whole body) vasospasm and increased systemic blood pressure, known as hypertension and, in pregnancy, as preeclampsia. Additionally, the increased systemic blood pressure leads to aldosterone production which causes water retention in the kidneys, and causes additional decreased kidney perfusion due to vasospasm, which causes the cycle of increased blood pressure regulation to continue.

To further illustrate the sensitivity of the left renal vein 114 to compressive abdominal forces, flow through a vein is equal to the pressure drop from one end of the vein to the other divided by the vascular resistance. In a vein, the vascular resistance is very sensitive to the diameter of the vein. The vascular resistance increases in inverse relation to the fourth power of radial decrease. For example, if the radius is halved, the vein pressure increases by a factor of sixteen in order to maintain constant blood flow through the vein. As a further example, if a vein is normally 6 mm in diameter and the diameter is decreased by 1 mm, the vascular pressure will double to maintain constant flow. Thus a slight compression of the left renal vein causing even a small change in diameter of the left renal vein can cause a substantial pressure increase within the vein to enable the constant flow to be maintained.

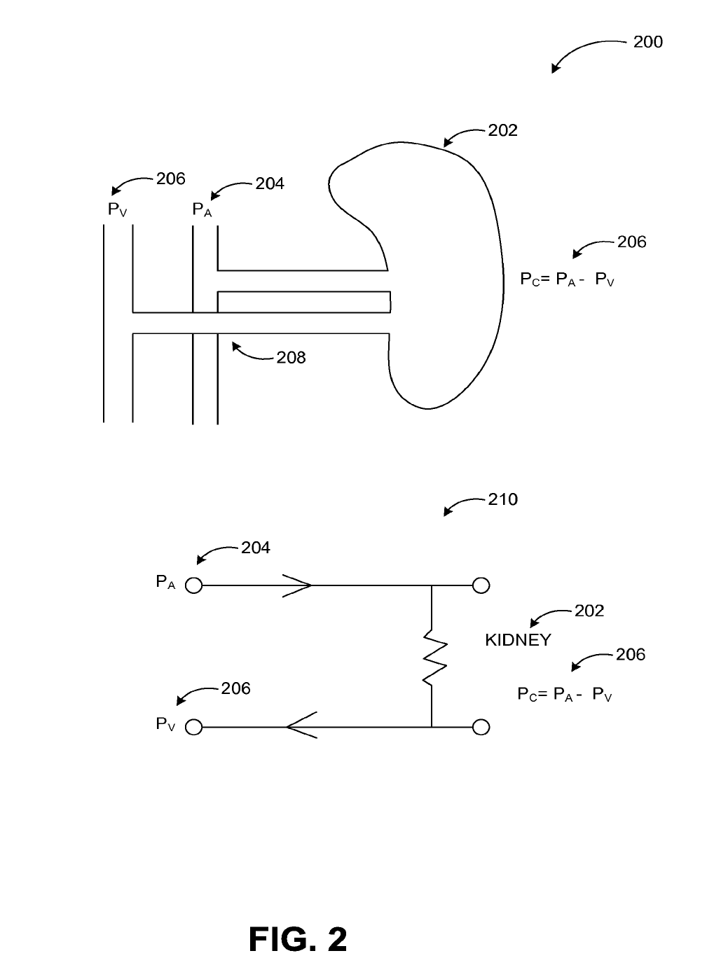

FIG. 2 illustrates an arrangement of the left renal vein and renal artery and a schematic of the corresponding blood pressures, arranged in accordance with at least some embodiments as described herein. As illustrated in diagram 200, in a typical blood pressure scenario when the left renal vein 208 is not compressed, the left kidney 202 observes the blood pressures in the aorta and the inferior vena cava. The blood pressure P_{c} 206 the left kidney 202 senses is the difference between the aortic blood pressure P_{A} 204 and the inferior vena cava blood pressure Pᵥ 206. (P_{c}=P_{A}−Pᵥ. Diagram 210 is a schematic representation of the blood pressure detected by the left kidney 202. The blood pressure P_{c} 206 the left kidney 202 sees is the difference between the aortic blood pressure P_{A} 204 and the inferior vena cava blood pressure Pᵥ 206. (P_{c}=P_{A}−Pᵥ).

FIG. 3 illustrates the location of the left renal vein where it crosses the aorta and a schematic diagram of the blood pressure when the left renal vein is compressed, arranged in accordance with at least some embodiments as described herein. As illustrated in diagram 300, if there is a pressure point 310 on the left renal vein 304 due to increased abdominal pressure P_{Ab} 302, it can compress the left renal vein 304 against the much harder aorta 308 resulting in a decreased diameter of the left renal vein 304. Pressure P_{z} is 306 the pressure needed in the left renal vein 304 to compensate for the compressive abdominal pressure P_{Ab} to allow blood to flow past the pressure point 310.

Diagram 310 illustrates a schematic layout of the pressures when there is increased abdominal pressure on the left renal vein 304. The pressure P_{c} 322 observed by the left kidney is P_{c}=P_{A}−P_{z}−Pᵥ. In this case Pc will be less than the case above where there is no compressive force on the LRV. While the aortic pressure P_{A} is the same in both models, the pressure the left kidney observes during compression of the left renal vein is less than what it should be and the kidney senses a low systematic blood pressure. When the left kidney senses a low systematic blood pressure, the left kidney begins an attempt to correct the low systematic blood pressure it senses by increasing the aortic pressure. Also illustrated in diagram 320 is an alternate collateral circulation return path for venous blood from the left kidney. This alternate or collateral circulation is common in about 85-90% of patients, and passes under the aorta. When the alternate collateral circulation is present, the present P_{z} will be zero such that the blood pressure P_{c} 322 observed by the left kidney is returned to P_{c}=P_{A}−Pᵥ, which represents the systemic blood pressure without pinching of the left renal vein due to increased abdominal pressure.

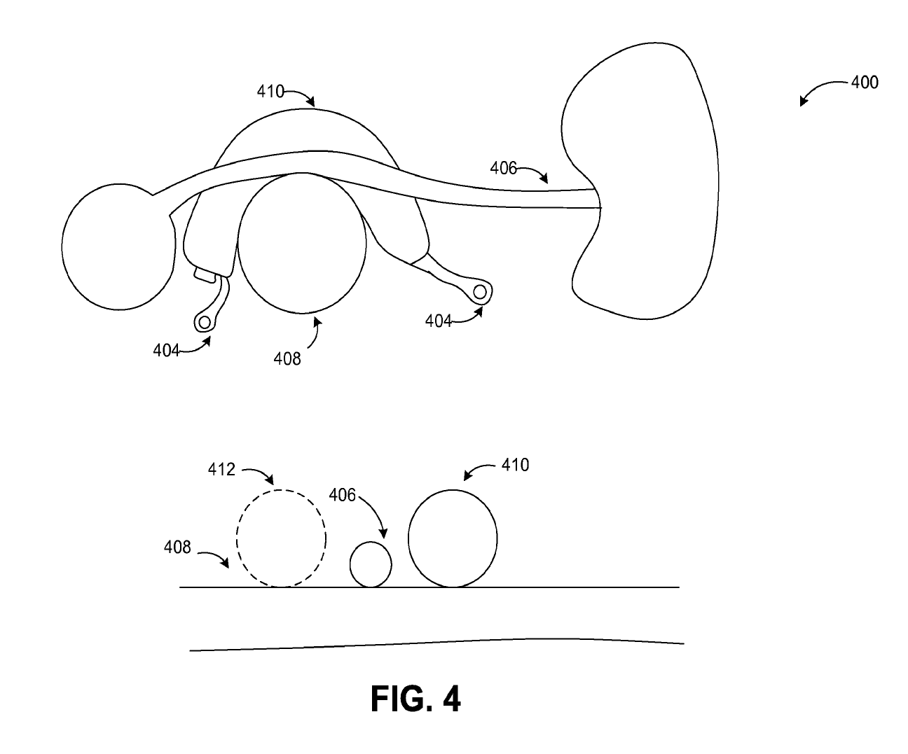

FIG. 4 illustrates a balloon for protecting the left renal vein from external pressures, arranged in accordance with at least some embodiments as described herein. As illustrated in diagram 400, the balloon 410 may be positioned next to the left renal vein 406 for preventing the left renal vein 406 from being compressed against the aorta 408 due to increased abdominal pressures. The balloon 410 may be positioned over the aorta 408 alongside the left renal vein 406 at the position where the aorta 408 crosses the left renal vein 406. The balloon 410 may be placed in an inferior or superior position to the left renal vein 406The balloon 410 may include anchoring sheaths 404 on each end of the balloon for enabling the balloon 410 to be attached in tissue within the abdominal cavity near the left renal vein 406. Anchoring the balloon 410 may provide stabilization for ensuring the balloon 410 remains in place with increased intra-abdominal pressures and forces.

The balloon 410 may be delivered and positioned in a deflated position and inflated to an inflated position. When the balloon 410 is in the inflated position, the balloon 410 may have an elongated cylindrical shape and a substantially circular cross-sectional shape. The balloon 410 may have a length in a range from about 6 cm to about 12 cm for enabling the balloon 410 to extend over the aorta from one side of the aorta to the other side. Additionally, the diameter of the balloon 410 may be larger than the diameter of the left renal vein 406, such that the balloon 410 may protect the left renal vein 406 from being compressed or pinched against the aorta 408. The diameter of the left renal vein 406 can vary in each individual, and a typical diameter of the left renal vein 406 may be in a range from about 6 mm to about 8 mm. The balloon 410 may have a diameter in a range from about 1 cm to about 1.5 cm such that the balloon 410 extends past the left renal vein 406 for protecting the left renal vein 406 from compression due to surrounding tissue and organs affected by the increased abdominal pressure.

In another example embodiment, two balloons may be used for providing more protection for the left renal vein 406. The first balloon 410 may be placed on one side of the left renal vein 406, and a second balloon 412 may be placed on the other side for preventing the left renal vein 406 from being compressed.

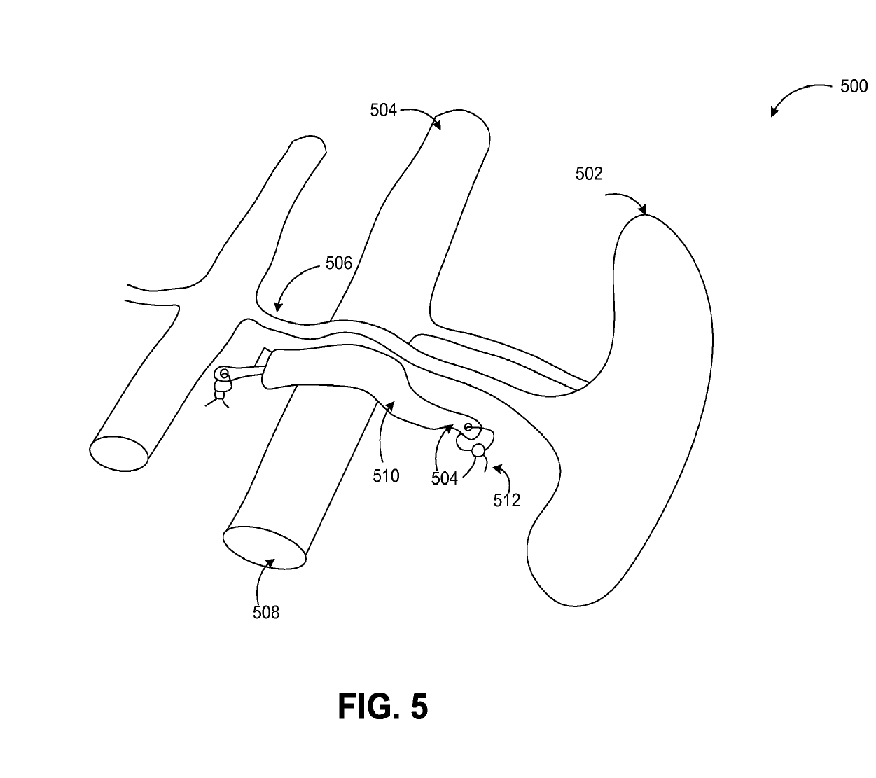

FIG. 5 illustrates a balloon anchored in position over the aorta and adjacent to the left renal vein, arranged in accordance with at least some embodiments as described herein. As demonstrated in diagram 500, a balloon 506 may be placed alongside a left renal vein 506 for preventing the left renal vein 506 from being compressed against the aorta 508 by organs and tissue shifting due to increased abdominal pressures. As previously described, the balloon 510 may be placed in an inferior or superior position to the left renal vein 506, such that it extends over and substantially perpendicular to the aorta 508. The balloon 510 may be composed from a noncompliant material configured to resist deformation when subjected to external pressure. In some examples the balloon 510 may be composed of a flexible polymer material such as polyethylene, polyamides (nylon), polypropylene, polyester, polyurethane, polystyrene, polysufone and/or polyethersulfone. In another example, the balloon 510 may be composed of a bio-absorbable material.

In an example embodiment, the balloon 510 may be delivered and positioned alongside the left renal vein 506 in a deflated position employing a laparoscopic technique. When the balloon has been placed in a desired position, the balloon 510 may be attached to the surrounding abdominal tissue and fascia. The balloon may include anchoring sheaths 504 on each end such for enabling the balloon 510 to be attached. The balloon 510 may be attached to the fascia using sutures 512 or other similar tissue attachment techniques to attach the sheaths 504 to the fascia. Example sutures 512 may be a bio-absorbable or non bio-absorbable material. Some example bio-absorbable materials include polyglycolic acid, polylactic acid, and polydioxanonc. Some non-bioabsorbable materials include polyamides (e.g. nylon) or polypropylene, for example. Once anchored in position, the balloon 510 be inflated such that the diameter of the balloon 510 is greater than the diameter of the left renal vein 506.

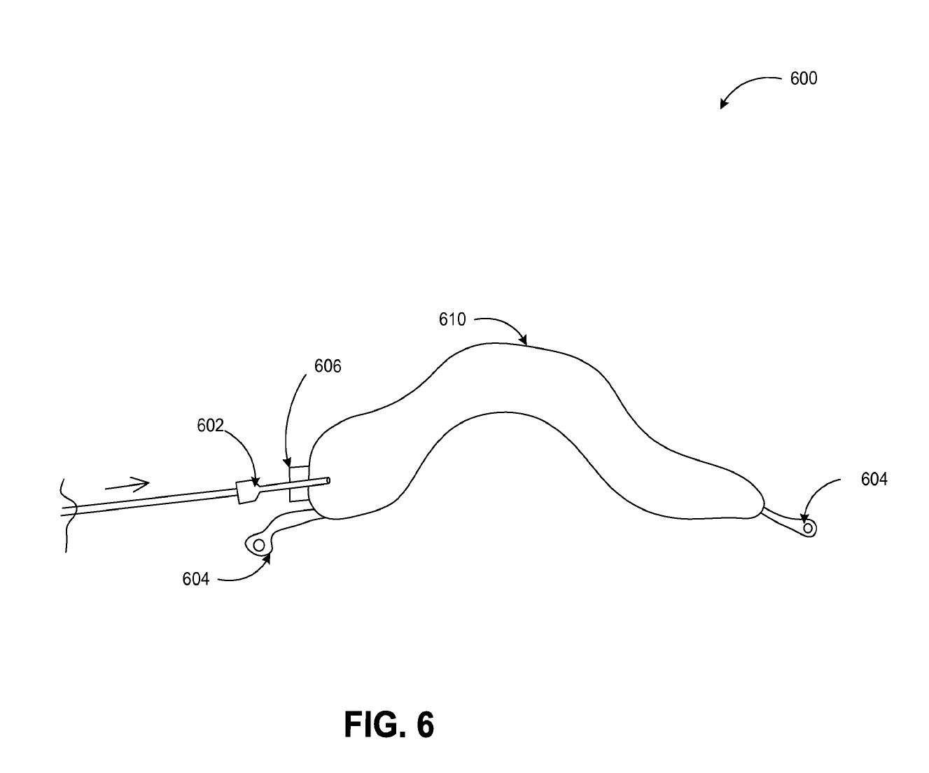

FIG. 6 illustrates inflation of a balloon employing a needle and port, arranged in accordance with at least some embodiments as described herein. As previously described, the balloon 610 may be delivered and positioned alongside the left renal vein in a deflated position employing a laparoscopic technique. Once anchored in position employing anchoring sheaths 604 on each end of the balloon 610, the balloon 610 may be inflated by filling the balloon with a sterile solution such as a saline solution. The saline solution may expand the balloon 610 such that the diameter of the balloon 610 is greater than the diameter of the left renal vein. When inflated, the balloon 610 may have a longitudinal shape that extends over the aorta in a substantially perpendicular orientation with respect to the aorta and in a substantially parallel orientation with respect to the left renal vein.

The balloon 610 may include a self-scaling port 606 for enabling insertion of a needle 602 for filling the balloon 610 with the saline solution. The needle 602 may be inserted into the self-sealing port 606 while the balloon 610 is outside the body, and the balloon 610 may be inserted and anchored in position while the needle 602 is inserted within the self-sealing port 606. After the balloon 610 is anchored in position, a syringe outside the body may be used to inflate the balloon 610 with the sterile saline solution. A physician can monitor the balloon 610 and the left renal vein to ensure the balloon 610 is protecting the left renal vein from compression during inflating. The balloon 610 may be inflated to resist an applied external pressure in a range from about 50 mmHg to about 100 mmHg, and when in the inflated position, the balloon 610 may be noncompliant such that the balloon may resist deformation and have very little change in volume when subjected to external pressure. Once inflated, the needle 602 may be removed and the self-sealing port 606 may seal to keep the saline solution within the balloon 610.

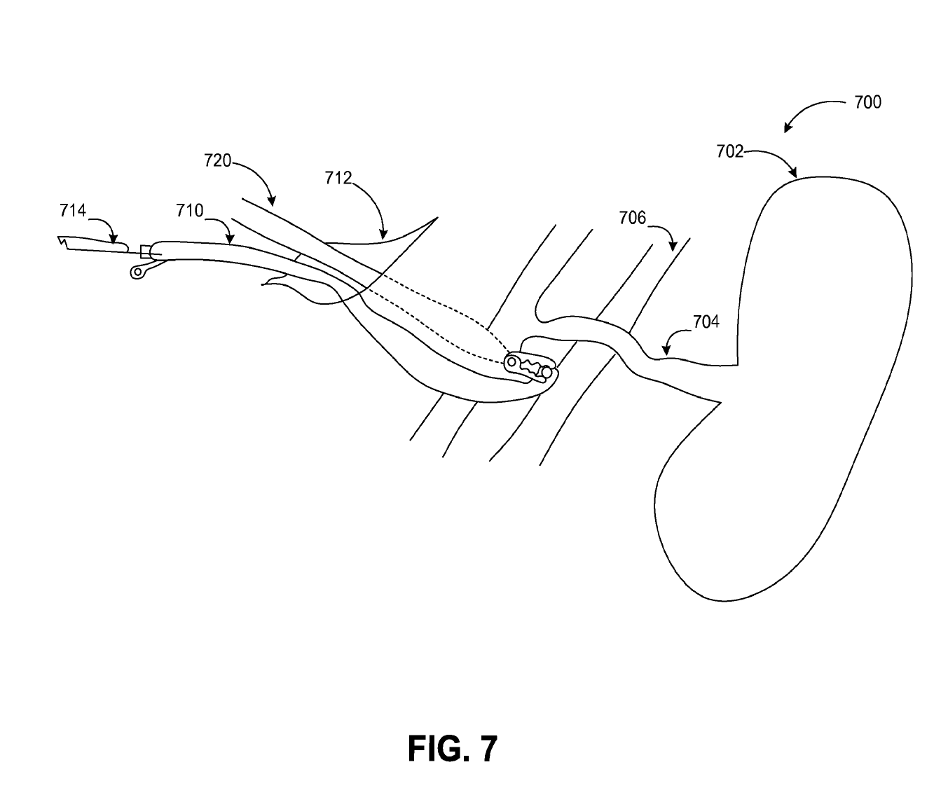

FIG. 7 illustrates delivery of a balloon employing laparoscopic tools, arranged in accordance with at least some embodiments as described herein. As illustrated in diagram 700, a balloon 710 may be inserted into the body and attached alongside a left renal vein 704 in the abdominal cavity near the aorta 706 employing a laparoscopic procedure. A delivery tube may provide percutaneous access to the abdominal cavity via at least one incision 712, and the balloon 710 may inserted through the incision 712. The balloon 710 may be inserted employing a laparoscopic guiding tool 720 attached to distal end of the balloon 710. The guiding tool 720 may be attached to an anchoring sheath at the distal end of the balloon 710. The balloon 710 may be delivered in an initial deflated position for enabling the incision 712 to remain small and for increasing the flexibility of the balloon 710 for delivery and anchoring in position alongside the left renal vein 704 and over the aorta 706. Additionally, a needle 714 may be inserted into the balloon at a self-sealing port on a proximal end of the balloon 710 outside the body before insertion.

When the balloon 710 has been placed into position employing the guiding tool 720, each end may be anchored into fascia employing the anchoring sheaths on each end of the balloon 710. Once anchored in position, the balloon 710 may be filled with the saline solution through the needle 714 for inflating to an inflated position. The needle 714 may be removed when the balloon is inflated to a desired size, and the self-sealing port may keep the saline solution within the balloon 710 and prevent leaking.

While embodiments have been discussed above using specific examples, components, and configurations, they are intended to provide a general guideline to be used for—providing an balloon for protecting the left renal vein from external pressures. These examples do not constitute a limitation on the embodiments, which may be implemented using other components, modules, and configurations using the principles described herein. Furthermore, actions discussed above may be performed in various orders, especially in an interlaced fashion.

According to some examples, the present disclosure describes a vascular reinforcement device for preventing compression of a blood vessel. The vascular reinforcement device may include a balloon having a substantially elongated shape configured to be placed adjacent to a wall of a blood vessel along a portion of the blood vessel and inflated for reducing an external pressure applied to the blood vessel when the balloon is in an inflated position. The vascular reinforcement device may include one or more sheaths attached to a first end and a second end of the balloon for anchoring the balloon to fascia surrounding the blood vessel to provide stabilization.

According to some examples, the balloon may be anchored to the fascia surrounding the blood vessel employing sutures coupled with the one or more sheaths. The sutures may be bio-absorbable. The sutures may be composed from one or more of polyglycolic acid, polylactic acid, and polydioxanone. The sutures may be composed of a non-absorbable material. The sutures may be composed of one or more of: nylon or polypropylene. The balloon may be composed of a noncompliant material configured to resist deformation when subjected to external pressure.

According to some examples, the balloon may be composed of a flexible polymer material. The polymer material may include one or more of polyamides, polyethylene, polypropylene, polyester, polyurethane, polystyrene, polysufone and/or polyethersulfone. The balloon may be composed of a bio-absorbable material. Some example bio-absorbable materials include polyglycolic acid, polylactic acid, and polydioxanone, or a copolymer of these.

According to some examples, the balloon may be configured to be expandable from a deflated position to the inflated position. The balloon in the inflated position may have a substantially circular cross-sectional shape with a diameter greater than a diameter of the adjacent blood vessel for reducing the external pressure applied to the blood vessel.

According to other examples, the balloon may be configured to be positioned adjacent to and substantially parallel to a left renal vein and over an aorta at a location where the aorta crosses the left renal vein. The balloon may be configured to be delivered in the deflated position employing a laparoscopic procedure. The balloon may be configured to be filled with a sterile solution for inflating to the inflated position.

According to some examples, the balloon may include a self-sealing port at one of a first end and a second end for enabling a needle to be inserted through the self-sealing port for filling the balloon with the sterile solution. The balloon in the inflated position may be configured to resist an applied external pressure from about 50 mmHg to about 100 mmHg.

The present disclosure also describes a method of preventing compression of a blood vessel from external bodily tissue forces. The method may include providing a vascular reinforcement device configured to resist external pressures from compressing a blood vessel, the vascular reinforcement device including an balloon having a substantially elongated shape, and one or more sheaths attached to a first end and a second end of the balloon for enabling the balloon to be anchored to fascia surrounding the blood vessel, and positioning the balloon adjacent to a wall of the blood vessel along a portion of the blood vessel such that when the balloon is in an inflated position, an external pressure applied to the blood vessel may be dampened.

According to some examples, the method may also include anchoring the balloon to the fascia surrounding the blood vessel employing sutures coupled with the one or more sheaths. The sutures may be one of bio-absorbable and non-absorbable. The method may also include constructing the balloon from a noncompliant material configured to resist deformation when subjected to the external pressure.

According to some examples, the balloon may be composed of a flexible polymer material. The balloon may be composed of a bio-absorbable material.

According to some examples, the method may also include configuring the balloon to be expandable from a deflated position to the inflated position. The method may also include configuring the balloon in the inflated position to have a substantially circular cross-sectional shape. The method may also include configuring the balloon in the inflated position to have a diameter greater than a diameter of an adjacent blood vessel for reducing external pressure applied to the blood vessel.

According to other examples, the method may also include configuring the balloon in the inflated position to have a diameter in the inflated position in a range from about 1 cm to about 1.5 cm. The method may also include positioning the balloon adjacent to and substantially parallel to a left renal vein and over an aorta at a location where the aorta crosses the left renal vein. The method may also include configuring the balloon to have a length in a range for enabling the balloon to extend over the aorta from one side of the aorta to the other side of the aorta.

According to further examples, the method may also include delivering the vascular reinforcement device with the balloon in the deflated position employing a laparoscopic procedure. The method may also include anchoring the balloon adjacent to the blood vessel while in the deflated position employing the laparoscopic procedure. The method may also include inflating the balloon by filling the balloon with a sterile solution employing a needle inserted into the balloon via a self-sealing port incorporated to one end of the balloon. The method may also include configuring the balloon in the inflated position to resist an applied external pressure of about 50 mmHg to about 100 mmHg.

According to further examples, the present disclosure describes a system for preventing compression of a vein from external bodily tissue forces. The system includes a surgical delivery tube for providing percutaneous access to an internal area of a body via at least one incision, and an balloon having a substantially elongated shape, wherein the balloon may be configured in an initial deflated position for delivery to the internal area of the body via the delivery tube and inflated to an inflated position at the internal area of the body, and one or more sheaths attached to a first end and a second end of the balloon for anchoring the balloon to fascia surrounding the blood vessel.

According to some examples, the internal area of the body may be a location in the abdominal cavity near the aorta. The balloon includes a self-sealing port for enabling a needle to be inserted through the self-sealing port for filling the balloon with a biocompatible sterile solution such as 0.9% saline. The balloon may be guided into the internal area of the body in the deflated position employing a guiding tool. The balloon may be anchored in position adjacent to the blood vessel while in the deflated position employing laparoscopic surgical instruments.

According to other examples, the balloon may be positioned in the deflated position adjacent to and substantially parallel to a left renal vein and over an aorta at a location where the aorta crosses the left renal vein. The balloon may be composed of a noncompliant material configured to resist deformation when subjected to external pressure. The balloon may be composed of a flexible polymer material including one or more of polyethylene, polyamides (nylon), polypropylene, polyester, polyurethane, polystyrene, polysufone and/or polyethersulfon. The balloon may be composed of a bio-absorbable material.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but arc not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A vascular reinforcement device for preventing compression of a blood vessel, comprising:
a balloon having a substantially elongated shape configured to be placed adjacent to a wall of a blood vessel along a portion of the blood vessel and inflated for reducing an external pressure applied to the blood vessel when the balloon is in an inflated position; and
one or more sheaths attached to a first end and a second end of the balloon for anchoring the balloon to fascia surrounding the blood vessel to provide stabilization.

2. (canceled)

3. The vascular reinforcement device of claim 1, wherein the balloon is anchored to the fascia surrounding the blood vessel employing sutures coupled with the one or more sheaths.

4. The vascular reinforcement device of claim 3, wherein the sutures are bio-absorbable, composed of one or more of polyglycolic acid, polylactic acid, and polydioxanone.

5. (canceled)

6. The vascular reinforcement device of claim 3, wherein the sutures are composed of a non-absorbable material selected from one or more of: polyamides or polypropylene.

7. (canceled)

8. The vascular reinforcement device of claim 1, wherein the balloon is composed of a noncompliant material configured to resist deformation when subjected to external pressure.

9. The vascular reinforcement device of claim 8, wherein the balloon is composed of a flexible polymer material selected from one or more of polyamides, polyethylene, polypropylene, polyester, polyurethane, polystyrene, polysufone and/or polyethersulfone.

10. (canceled)

11. The vascular reinforcement device of claim 1, wherein the balloon is composed of a bio-absorbable material selected from one or more of polyglycolic acid, polylactic acid, and polydioxanone.

12. The vascular reinforcement device of claim 1, wherein the balloon is configured to be expandable from an initial deflated position to the inflated position.

13-18. (canceled)

19. A method of preventing compression of a blood vessel from external bodily tissue forces, comprising:
providing a vascular reinforcement device configured to resist external pressures from compressing a blood vessel, the vascular reinforcement device including a balloon having a substantially elongated shape, and one or more sheaths attached to a first end and a second end of the balloon for enabling the balloon to be anchored to fascia surrounding the blood vessel; and
positioning the balloon adjacent to a wall of the blood vessel along a portion of the blood vessel such that when the balloon is in an inflated position, an external pressure applied to the blood vessel is dampened.

20-24. (canceled)

25. The method of claim 19, further comprising:
configuring the balloon to be expandable from a deflated position to the inflated position.

26. The method of claim 25, further comprising:
configuring the balloon in the inflated position to have a substantially circular cross-sectional shape.

27. The method of claim 25, further comprising:
configuring the balloon in the inflated position to have a diameter greater than a diameter of an adjacent blood vessel for reducing external pressure applied to the blood vessel.

28. The method of claim 19, further comprising:
positioning the balloon adjacent to and substantially parallel to a left renal vein and over an aorta at a location where the aorta crosses the left renal vein.

29. The method of claim 28, further comprising:
configuring the balloon to have a length in a range for enabling the balloon to extend over the aorta from one side of the aorta to the other side of the aorta.

30. The method of claim 19, further comprising:
delivering the balloon in the deflated position employing a laparoscopic procedure.

31. The method of claim 30, further comprising:
anchoring the balloon adjacent to the blood vessel while in the deflated position employing the laparoscopic procedure.

32. The method of claim 31, further comprising:
inflating the balloon by filling the balloon with a sterile solution employing a needle inserted into the balloon via a self-sealing port incorporated to one end of the balloon.

33. (canceled)

34. A system for preventing compression of a vein from external bodily tissue forces, comprising:
a surgical delivery tube for providing percutaneous access to an internal area of a body via at least one incision; and
a balloon having a substantially elongated shape, wherein the balloon is configured in an initial deflated position for delivery to the internal area of the body via the delivery tube and inflated to an inflated position at the internal area of the body; and
one or more sheaths attached to a first end and a second end of the balloon for anchoring the balloon to fascia surrounding the blood vessel.

35. The system of claim 34, wherein the internal area of the body is a location in the abdominal cavity near an aorta.

36. The system of claim 35, wherein the balloon includes a self-sealing port enabling a needle to be inserted through the self-sealing port for filling the balloon with a sterile solution.

37. The system of claim 35, wherein the balloon is guided into the internal area of the body in the deflated position employing a guiding tool.

38. The system of claim 37, wherein the balloon is anchored in position adjacent to the blood vessel while in the deflated position employing laparoscopic surgical instruments.

39. The system of claim 37, wherein the balloon is positioned in the deflated position adjacent to and substantially parallel to a left renal vein and over the aorta at a location where the aorta crosses the left renal vein.

40-42. (canceled)
